(19) United States
(12) Patent Application Publication
(10) Pub. No.: US 2025/0229050 A1
(43) Pub. Date: Jul. 17, 2025

(54) Oxygen Generator with Negative Oxygen Ions and Diffused Oxygen System

(71) Applicant: Xizang Fuyang Technology Co., Ltd., Lhasa (CN)

(72) Inventors: Loufei LIN, Lhasa (CN); Jie LIN, Lhasa (CN)

(73) Assignee: Xizang Fuyang Technology Co., Ltd., Lhasa (CN)

(21) Appl. No.: 19/017,268

(22) Filed: Jan. 10, 2025

(30) Foreign Application Priority Data
Jan. 11, 2024 (CN) 202420127653.5

(51) Int. Cl. A61M 16/10; A61M 16/20

(52) U.S. Cl. CPC A61M 16/101; A61M 16/202; A61M 2016/1025; A61M 2016/103; A61M 2202/0208

(57) ABSTRACT

Disclosed in the present invention is an oxygen generator with negative oxygen ions and the diffused oxygen system. The oxygen generator comprising an equipment housing, a control circuit board, a power supply device, a compressor and a plurality of molecular sieve assembly arranged in the equipment housing. It divides the compressed gas output by the compressor into two paths, one of which is used to prepare oxygen, and the other is directly used for the gas-excited negative oxygen ion generator to prepare negative oxygen ions. In this way, the negative oxygen ion generator have sufficient pressure gas to prepare a sufficient amount of negative oxygen ions. In addition, the oxygen interface of the oxygen generator is not occupied, and the pressure of the oxygen interface is not limited, thereby meeting various oxygen needs such as oxygen inhalation.

### RELATED APPLICATIONS

The present patent document claims the benefit of priority to Patent Application No. 202420127653.5, filed Jan. 11, 2024, and entitled “An Oxygen Generator with Negative Oxygen Ions and Diffused Oxygen System,” the entire contents of each of which are incorporated herein by reference.

### BACKGROUND

### 1. Technical Field

The present invention relates to the field of air conditioning equipment, and in particular to an oxygen generator with negative oxygen ions and diffused oxygen system.

### 2. Background Information

Life cannot do without oxygen, and the health and therapeutic effects of oxygen inhalation have been widely recognized and applied by the medical community. Small molecular sieve oxygen generators using PSA pressure swing adsorption have entered tens of millions of households as medical devices and home health care equipment. Small molecular sieve oxygen generators can extract oxygen with a concentration of about 93% from the air by simply turning on the power, and provide it to users at a flow rate of 1-10 L/minute, which can improve the body's blood oxygen saturation, improve tissue hypoxia, promote the body's metabolic function, and maintain the body's life activities. It is an important treatment method. Long-term use of oxygen therapy helps to reduce hypoxemia, relieve pulmonary hypertension, relieve bronchospasm, improve the patient's physical condition, improve sleep and brain function, improve exercise endurance and quality of life, improve chronic obstructive pulmonary disease, and prolong life.

Negative oxygen ions in the air are a kind of negatively charged air particles. Just like vitamins in food, they have a very important impact on human life activities. Some people call them “air vitamins” and some even call them “longevity factors”. In nature, although atmospheric ions are invisible and intangible, people can feel the existence of negative oxygen ions. In nature, negative oxygen ions can grow or disappear, maintaining a certain dynamic equilibrium state. Under normal circumstances, the concentration of negative oxygen ions on the ground surface is about 400-700/cm3. Similarly, the high and low concentrations of negative oxygen ions are closely related to people's health. People need it all the time, especially in today's increasingly serious pollution. According to research by environmental experts, when the concentration of negative oxygen ions in the air is below 20 per cubic centimeter, people will feel tired, dizzy, uncomfortable, and even dizzy, headache, nausea, vomiting, emotional anxiety, breathing difficulties, decreased work efficiency, and cause some unknown symptoms. Pathological changes. When the number reaches 1000-10000 per cubic centimeter, people will feel calm and peaceful. When the number reaches more than 10,000 per cubic centimeter, people will feel refreshed, comfortable and cozy. When the number reaches more than 100,000 per cubic centimeter of air, it can calm down, stop wheezing, eliminate fatigue, regulate nerves and have the effect of preventing and curing diseases.

In order to provide oxygen and negative oxygen ions at the same time, people combine negative oxygen ion generators with oxygen concentrators. For example, New-style Practical Patents of China CN208274806U provides a negative oxygen ion oxygen concentrator, including an oxygen concentrator body, an oxygen supply pipe, a humidification bottle filled with humidification liquid and an oxygen output pipe connected in sequence. The humidification bottle is provided with an airflow impact type negative oxygen ion generator, and the airflow impact type negative oxygen ion generator includes a body, a humidification bottle oxygen inlet pipe installed on the top of the body, and the air inlet end of the humidification bottle oxygen inlet pipe is connected to the oxygen supply pipe and installed on the top of the humidification bottle; an impact chamber is provided in the body, and the impact type penetrates the body to connect with the humidification liquid, and at least one impact hole is opened on the two opposite inner walls of the impact chamber, and the impact hole connects the humidification bottle oxygen inlet pipe and the impact chamber, so as to accelerate the oxygen flow into the impact chamber, collide or collide with the humidification liquid and the wall in the impact chamber, and generate high concentration of ecological grade negative oxygen ions.

The existing combination of an gas-excited negative oxygen ion generator and an oxygen concentrator usually involves directly connecting oxygen to the gas-excited negative oxygen ion generator and combining the gas-excited negative oxygen ion generator with a humidification bottle. Since the gas-excited negative oxygen ion generator requires a higher air pressure and gas volume, when oxygen is directly connected to the gas-excited negative oxygen ion generator, nasal oxygen inhalation is not possible. If the oxygen is diverted for use, there is a defect of insufficient gas volume. Because the existing portable oxygen concentrators usually have an oxygen output of 10 L or 5 L, the oxygen output is not enough to prepare enough negative oxygen ions, and the existing oxygen concentrators need to be further optimized.

### BRIEF SUMMARY

A technical problem to be solved by the present invention is to provide an oxygen generator with negative oxygen ions and diffused oxygen system, aiming to solve the technical problems of insufficient gas volume and single function faced by the oxygen concentrator combining a gas-excited negative oxygen ion generator with a humidification bottle.

In order to solve the above technical problem, the technical solution of the present invention is as follows:

The first aspect of the present invention provides an oxygen concentrator with negative oxygen ions, including an equipment housing, a control circuit board, a power supply device, a compressor and a plurality of molecular sieve assembly arranged in the equipment housing, wherein the input end of the compressor is connected to an exhaust interface, and the output end of the compressor divides the compressed gas into two paths, one of which is connected to the molecular sieve assembly through an electromagnetic reversing valve, and the molecular sieve assembly inputs oxygen into the oxygen interface; the other compressed gas is connected to an gas-excited negative oxygen ion generator, and the gas-excited negative oxygen ion generator is used to provide negative oxygen ions.

Further, the oxygen generator includes a diverter valve, the output end of the compressor is connected to the diverter valve, and the compressed gas is divided into two paths through the diverter valve, one of which is connected to the molecular sieve assembly through an electromagnetic reversing valve, and the other compressed gas is connected to an gas-excited negative oxygen ion generator through a pressure gas interface.

Further, a gas volume regulating valve or a limiting hole is also provided at the input end of the gas-excited negative oxygen ion generator to control the amount of gas input into the gas-excited negative oxygen ion generator.

Further, the oxygen generator also includes an oxygen storage tank disposed in the device housing, the molecular sieve assembly inputs oxygen into the oxygen storage tank, and the oxygen storage tank is connected to an oxygen interface on one side of the device housing.

Optionally, the device housing is provided with a display screen and a plurality of control buttons, and the display screen and the control buttons are connected to the control circuit board. Specifically, the display screen may be one of an LED display screen, a liquid crystal display screen and a touch display screen. The control buttons may be capacitive buttons, mechanical buttons or touch buttons. The display screen and the control buttons can clearly display various parameters of the oxygen concentrator operation and perform control operations, which is convenient for users to use.

Specifically, an exhaust disinfection assembly and a muffler are also provided corresponding to the electromagnetic reversing valve. Optionally, a frequency conversion controller is connected or integrated with one of the control circuit board and the power supply device, and the speed of the compressor is adjusted by the frequency conversion controller, so that the amount of extracted gas, the amount of oxygen input and the oxygen concentration can be adjusted.

Further, an oxygen pressure regulating valve and an oxygen sensor are provided at the output end of the oxygen storage tank. The oxygen sensor is arranged on the oxygen pipeline between the oxygen storage tank and the oxygen interface. The output end of the oxygen sensor is connected to the oxygen interface through a flow regulating valve, and the oxygen sensor is connected to the control circuit board.

Specifically, a bacteria filter and an oxygen check valve are sequentially connected between the output end of the oxygen sensor and the flow regulating valve.

Optionally, the oxygen sensor is an ultrasonic oxygen sensor, which is used to collect oxygen concentration data and oxygen flow data in the oxygen pipeline. Ultrasonic oxygen sensor is a sensor used to measure gas flow and oxygen concentration in binary gas. It adopts ultrasonic detection technology and is superior to electrochemical and other oxygen sensors. It has functions such as numerical display, online monitoring, and status alarm, and can be widely used in household and medical oxygen concentrators, oxygen concentrators, and other occasions.

Optionally, the control circuit board is connected with a wireless communication device, and the wireless communication device is one or more of a Bluetooth communication module, a wireless RF communication module, a cellular network communication module, and a Wi-Fi communication module. Preferably, it is a Bluetooth 4.0 communication module, which realizes data communication with a mobile terminal, and can be connected to a network server through a mobile terminal or directly connected to a network server.

The second aspect of the present invention provides a diffused oxygen system, comprising an oxygen generator, an intelligent controller arranged in the oxygen-demanding space, and a gas-excited negative oxygen ion generator, wherein the oxygen generator comprises an equipment housing, a control circuit board, a power supply device, a compressor, and a plurality of molecular sieve assembly arranged in the equipment housing, wherein the input end of the compressor is connected to an exhaust interface, and the input end of the compressor is connected to the oxygen-demanding space through the exhaust interface to extract gas, or extracts gas from the oxygen-demanding space and the external space simultaneously;
The output end of the compressor divides the compressed gas into two paths, one of which is connected to the molecular sieve assembly through an electromagnetic reversing valve, and the molecular sieve assembly inputs oxygen into the oxygen interface, and the oxygen interface inputs oxygen to the air outlet of the intelligent controller through a connecting pipeline; the other path is connected to the gas-excited negative oxygen ion generator through a connecting pipeline, and the gas-excited negative oxygen ion generator is arranged on the connecting pipeline outside or inside the oxygen-demanding space, and the output end of the gas-excited negative oxygen ion generator is connected to the air outlet of the intelligent controller, or the input end of the gas-excited negative oxygen ion generator is arranged at the air outlet of the intelligent controller, and the output end of the gas-excited negative oxygen ion generator is directly connected to the oxygen-demanding space.

Further, the oxygen generator is arranged outside the oxygen-demanding space, the exhaust interface of the compressor is connected to the oxygen-demanding space through an gas extraction pipeline, and all or part of the air in the oxygen-demanding space is extracted to produce oxygen, and external air is used to replenish the oxygen-demanding space for air balance.

Further, the oxygen generator is arranged in the oxygen-demanding space, and the exhaust interface of the compressor is connected to the oxygen-demanding space to extract gas, or is connected to the external space through an gas extraction pipeline and extracts gas from the oxygen-demanding space and the external space at the same time; the oxygen generator discharges nitrogen to the outside of the oxygen-demanding space through a nitrogen gas extraction pipeline.

Further, the intelligent controller is arranged in the oxygen-demanding space, including a sensor group for real-time monitoring of air state parameters and data exchange with a host computer. The sensor group is connected to or integrally arranged with the intelligent controller, and is used to detect the real-time oxygen concentration value and the real-time carbon dioxide concentration value in the oxygen-demanding space according to a preset period. When the real-time oxygen concentration value or the real-time carbon dioxide concentration value is abnormal, the operating parameters of the oxygen generator are queried and detected and an alarm is issued.

The intelligent controller further includes a processor, a network communication device connected to the processor, a working voltage device, an indoor display screen, and an intelligent control key group.

With the above-mentioned technical solution, the oxygen generator with negative oxygen ions and diffused oxygen system divide the compressed gas output by the compressor into two paths, one of which is used to prepare oxygen, and the other is directly used for the gas-excited negative oxygen ion generator to prepare negative oxygen ions. In this way, the negative oxygen ion generator have sufficient pressure gas to prepare a sufficient amount of negative oxygen ions. In addition, the oxygen interface of the oxygen generator is not occupied, and the pressure of the oxygen interface is not limited, thereby meeting various oxygen needs such as oxygen inhalation.

### BRIEF DESCRIPTION OF THE DRAWINGS

In order to explain the technical solutions in the embodiments or the prior art of the present invention more clearly, a brief description will be given below with reference to the accompanying drawings which are used in the description of the embodiments or the prior art, and it is obvious that the drawings in the description below are merely some embodiments of the present invention, and it would have been obvious for a person skilled in the art to obtain other drawings according to these drawings without involving any inventive effort.

FIG. 1 is a schematic diagram showing the principle of an oxygen generator with negative oxygen ions according to the present invention;

FIG. 2 is a second schematic diagram of the oxygen generator with negative oxygen ions of the present invention;

FIG. 3 is a front view structural diagram of a first oxygen generator with negative oxygen ions according to the present invention;

FIG. 4 is a side view of the structure of a second oxygen generator with negative oxygen ions according to the present invention;

FIG. 5 is a structural diagram of a first diffused oxygen system according to an embodiment of the present invention;

FIG. 6 is a structural diagram of a second diffused oxygen system according to an embodiment of the present invention;

FIG. 7 is a functional block diagram of an intelligent controller according to an embodiment of the present invention;

In the figure, 10—an oxygen generator with negative oxygen ions, 20—gas-excited negative oxygen ion generator, 30—humidification bottle, 40—intelligent controller, 50—connecting pipeline, 60—gas extraction pipeline, 70—nitrogen gas extraction pipeline, 80—oxygen-demanding space;
11—control circuit board, 12—power supply device, 13—compressor, 14—diverter valve, 15—electromagnetic reversing valve, 16—molecular sieve assembly, 17—oxygen storage tank, 18—oxygen pressure regulating valve, 19—oxygen sensor, 110—bacteria filter, 111—oxygen check valve, 112—flow regulating valve, 113—oxygen interface, 114—muffler, 115—exhaust disinfection assembly, 116—power-off alarm device, 117—wireless communication device, 118—control button, 119—display screen, 120—speaker, 121—pressure gas interface, 122—exhaust interface, 123—gas volume regulating valve (limiting hole);
41—processor, 42—sensor group, 43—network communication device, 44—working voltage device, 45—indoor display screen, 46—intelligent control key group.

### DETAILED DESCRIPTION OF THE DRAWINGS AND THE PRESENTLY PREFERRED EMBODIMENTS

Reference will now be made in detail to the embodiments of the present invention, examples of which are illustrated in the accompanying drawings. It is to be understood that the description of these embodiments is intended to aid in the understanding of the present invention, and is not intended to limit the scope of the present invention. Further, the technical features involved in the various embodiments of the present invention described below can be combined with each other as long as they do not conflict with each other.

### Embodiment 1

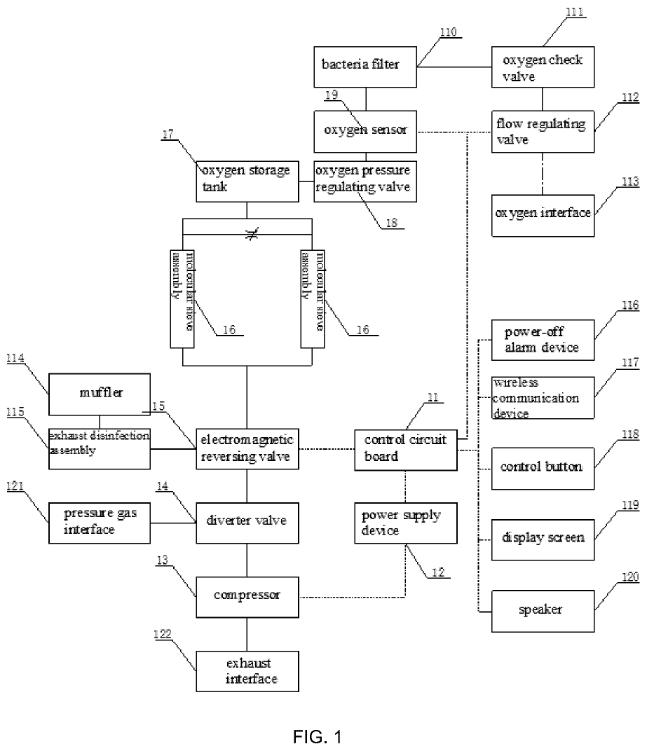

As shown in FIGS. 1, an embodiment of the present invention provides an oxygen generator with negative oxygen ions and diffused oxygen system 10, including an equipment housing, a control circuit board 11, a power supply device 12, a compressor 13 and a plurality of molecular sieve assembly 16 arranged in the equipment housing, the input end of the compressor 13 is connected to the exhaust interface 121, and the output end of the compressor 13 divides the compressed gas into two paths, one of which is connected to the molecular sieve assembly 16 through an electromagnetic reversing valve 15, and the molecular sieve assembly 16 inputs oxygen into the oxygen interface 113; the other compressed gas is connected to an gas-excited negative oxygen ion generator 20, and the gas-excited negative oxygen ion generator 20 is used to provide negative oxygen ions.

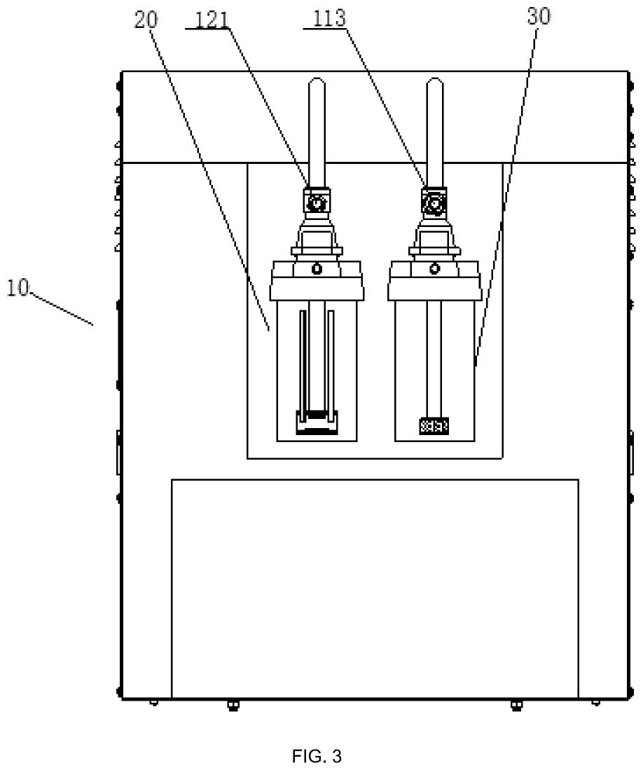

As shown in FIGS. 1 and 3, the oxygen concentrator 10 includes a diverter valve 14, and the output end of the compressor 13 is connected to the diverter valve 14. The compressed gas is divided into two paths through the diverter valve 14, one of which is connected to the molecular sieve assembly 16 through an electromagnetic reversing valve 15, and the other compressed gas is connected to a gas-excited negative oxygen ion generator 20 through a pressure gas interface 121.

Optionally, the diverter valve 14 can be an electromagnetic diverter regulating valve, which divides the amount of compressed gas from the compressor 13 into two paths according to the proportion of the control instruction, or the diverter valve 14 is replaced by a three-way joint, and the compressed gas is input through the three-way joint in one path, and the two paths are diverted and output.

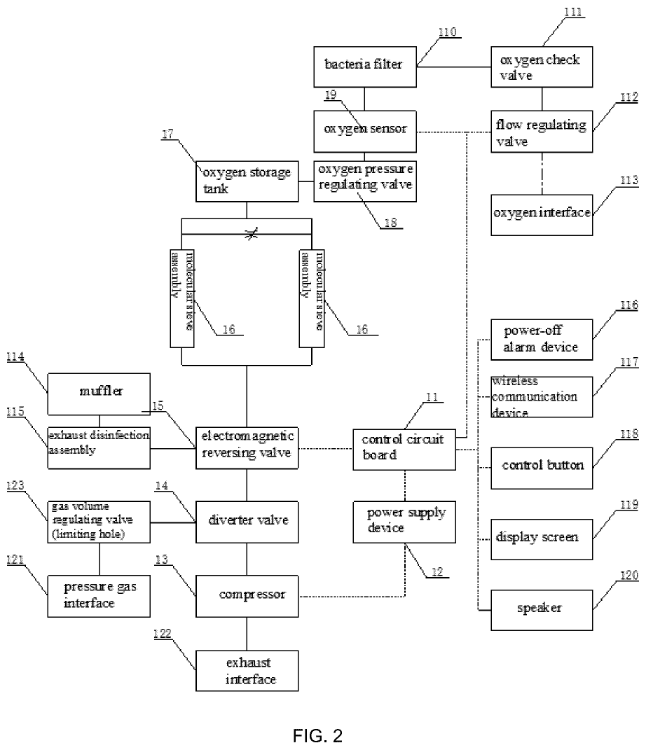

As shown in FIGS. 2 and 3, the input end of the gas-excited negative oxygen ion generator 20 is also provided with a gas volume regulating valve or a limiting hole 123 for controlling the amount of gas input into the gas-excited negative oxygen ion generator 20.

As shown in FIG. 1, the oxygen generator 10 further includes an oxygen storage tank 17 disposed in the device housing. The molecular sieve assembly 16 inputs oxygen into the oxygen storage tank 17, and the oxygen storage tank 17 is connected to an oxygen interface 113 on one side of the device housing.

Optionally, the device housing is provided with a display screen 119 and a plurality of control buttons 118, and the display screen 119 and the control buttons 118 are connected to the control circuit board 11. Specifically, the display screen 119 can be one of an LED display screen, a liquid crystal display screen and a touch display screen. The control button 118 can be a capacitive button, a mechanical button or a touch button. The display screen 119 and the control button 118 can clearly display various parameters of the oxygen concentrator 10 and perform control operations, which is convenient for users to use.

Specifically, an exhaust disinfection assembly 115 and a muffler 114 are also provided corresponding to the electromagnetic reversing valve 15. Optionally, a frequency conversion controller is connected or integrated with one of the control circuit board 11 and the power supply device 12, and the speed of the compressor 13 is adjusted by the frequency conversion controller, so that the amount of extracted gas, the amount of oxygen input and the oxygen concentration can be adjusted.

As shown in FIGS. 1, the output end of the oxygen storage tank 17 is provided with an oxygen pressure regulating valve 18 and an oxygen sensor 19. The oxygen sensor 19 is arranged on the oxygen pipeline between the oxygen storage tank 17 and the oxygen interface 113. The output end of the oxygen sensor 19 is connected to the oxygen interface 113 through a flow regulating valve 112, and the oxygen sensor 19 is connected to the control circuit board 11.

Specifically, a bacteria filter 110 and an oxygen check valve 111 are sequentially connected between the output end of the oxygen sensor 19 and the flow regulating valve 112.

Optionally, the oxygen sensor 19 is an ultrasonic oxygen sensor, which is used to collect oxygen concentration data and oxygen flow data in the oxygen pipeline. Ultrasonic oxygen sensor, a sensor for measuring gas flow and oxygen concentration in binary gas, adopts ultrasonic detection technology, which is superior to electrochemical and other oxygen sensors; it has functions such as numerical display, online monitoring, status alarm, etc., and can be widely used in household and medical oxygen concentrators, oxygen concentrators and other occasions.

Optionally, the control circuit board 11 is connected to a speaker 120 for providing sound prompts to the user. The control circuit board 11 is integrated with a power-off alarm device 116, which emits sound and light prompts when the device is powered off to remind the user to take necessary measures to avoid harm to the user.

Optionally, the control circuit board 11 is connected to a wireless communication device 117, and the wireless communication device 117 is one or more of a Bluetooth communication module, a wireless RF communication module, a cellular network communication module, and a Wi-Fi communication module. Preferably, it is a Bluetooth 4.0 communication module to achieve data communication with a mobile terminal, and can be connected to a network server through a mobile terminal or directly connected to a network server.

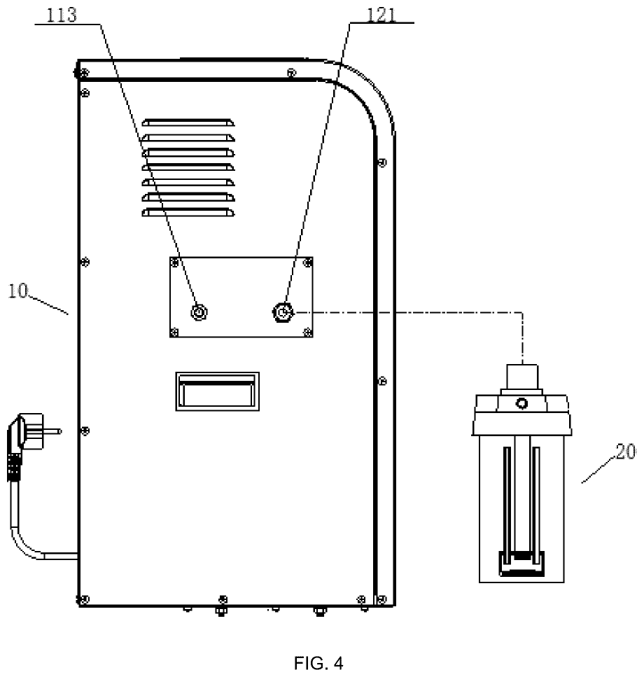

As shown in FIGS. 3 and 4, due to the different specifications of the oxygen generator, the gas-excited negative oxygen ion generator 20 and the humidification bottle 30 can be integrated into the device housing, or connected to the pressure gas interface 121 and the oxygen interface 113 on the device housing through connecting pipelines.

### Embodiment 2

As shown in FIGS. 1, 5 and 6, an embodiment of the present invention provides a diffused oxygen system, comprising an oxygen generator 10, an intelligent controller 40 disposed in the oxygen-demanding space 80 and a gas-excited negative oxygen ion generator 20, wherein the oxygen generator 10 comprises an equipment housing, a control circuit board 11, a power supply device 12, a compressor 13 and a plurality of molecular sieve assembly 16 disposed in the equipment housing, the input end of the compressor 13 being connected to an exhaust interface 122, the input end of the compressor 13 being connected to the oxygen-demanding space 80 through the exhaust interface 122 to extract gas, or extracting gas from the oxygen-demanding space 80 and the external space simultaneously;

The output end of the compressor 13 divides the compressed gas into two paths, one of which is connected to the molecular sieve assembly 16 through an electromagnetic reversing valve 15, and the molecular sieve assembly 16 inputs oxygen into the oxygen interface 113, and the oxygen interface 113 inputs oxygen to the air outlet of the intelligent controller 40 through the connecting pipeline 50; the other path is connected to the gas-excited negative oxygen ion generator 20 through the connecting pipeline 50, and the gas-excited negative oxygen ion generator 20 is arranged on the connecting pipeline 50 on the outside or inside of the oxygen-demanding space 80, and the output end of the gas-excited negative oxygen ion generator 20 is connected to the air outlet of the intelligent controller 40, or the input end of the gas-excited negative oxygen ion generator 20 is arranged at the air outlet of the intelligent controller 40, and the output end of the gas-excited negative oxygen ion generator 20 is directly connected to the oxygen-demanding space 80.

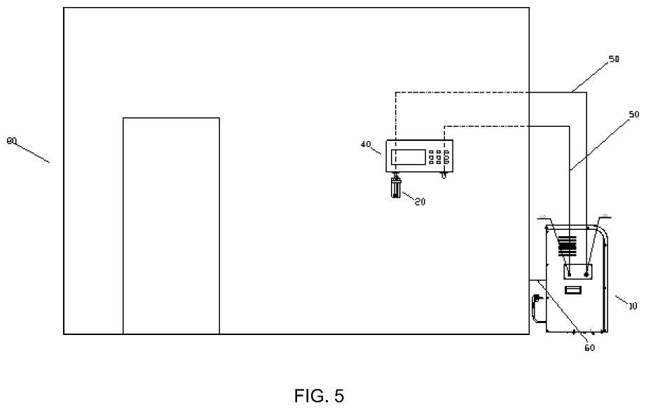

As shown in FIG. 5, the oxygen generator 10 is arranged outside the oxygen-demanding space 80, and the exhaust interface 122 of the compressor 13 is connected to the oxygen-demanding space 80 through the gas extraction pipeline 60, and all or part of the air in the oxygen-demanding space 80 is extracted to produce oxygen, and the external air is used to replenish the oxygen-demanding space 80 for air balance.

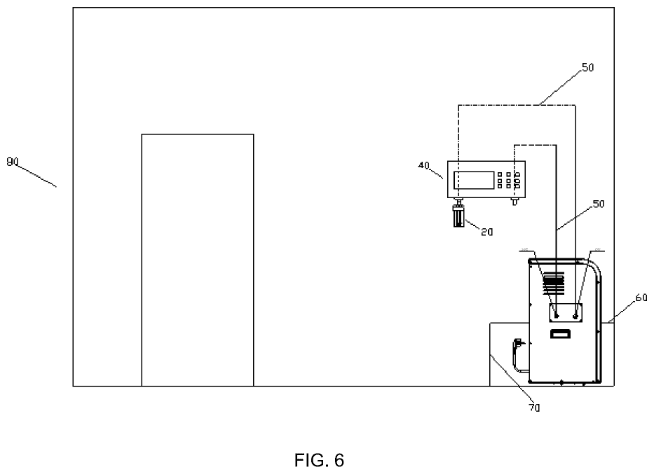

As shown in FIGS. 6, the oxygen generator 10 is disposed in the oxygen-demanding space 80, and the exhaust interface 122 of the compressor 13 is connected to the oxygen-demanding space 80 to extract gas, or is connected to the external space through a gas extraction pipeline 60 and extracts gas from the oxygen-demanding space 80 and the external space simultaneously; the oxygen generator 10 discharges nitrogen to the outside of the oxygen-demanding space 80 through a nitrogen gas extraction pipeline 70.

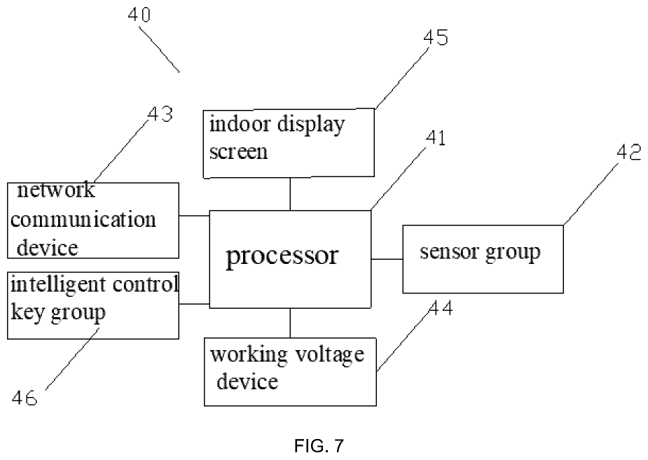

As shown in FIGS. 7, the intelligent controller 40 is arranged in the oxygen-demanding space 80, including a sensor group 42 for real-time monitoring of air state parameters and data exchange with a host computer. The sensor group 42 is connected to or integrally arranged with the intelligent controller 40, and is used to detect the real-time oxygen concentration value and the real-time carbon dioxide concentration value in the oxygen-demanding space 80 according to a preset period. When the real-time oxygen concentration value or the real-time carbon dioxide concentration value is abnormal, the operating parameters of the oxygen generator 10 are queried and detected and an alarm is issued.

The intelligent controller 40 further includes a processor 41, a network communication device 43 connected to the processor, a working voltage device 44, an indoor display screen 45, and an intelligent control key group 46.

The of the embodiment of the present invention divide the compressed gas output by the compressor into two paths, one of which is used to prepare oxygen, and the other is directly used for the gas-excited negative oxygen ion generator to prepare negative oxygen ions. In this way, the negative oxygen ion generator have sufficient pressure gas to prepare a sufficient amount of negative oxygen ions. In addition, the oxygen interface of the oxygen generator is not occupied, and the pressure of the oxygen interface is not limited, thereby meeting various oxygen needs such as oxygen inhalation.

1. A oxygen concentrator with negative oxygen ions, comprising an equipment housing, a control circuit board, a power supply device, a compressor and a plurality of molecular sieve assembly arranged in the equipment housing, wherein the input end of the compressor is connected to an exhaust interface, and the output end of the compressor divides the compressed gas into two paths, one of which is connected to the molecular sieve assembly through an electromagnetic reversing valve, and the molecular sieve assembly inputs oxygen into the oxygen interface; the other compressed gas is connected to an gas-excited negative oxygen ion generator, and the gas-excited negative oxygen ion generator is used to provide negative oxygen ions.

2. The oxygen concentrator with negative oxygen ions according to claim 1, wherein the oxygen generator includes a diverter valve, the output end of the compressor is connected to the diverter valve, and the compressed gas is divided into two paths through the diverter valve, one of which is connected to the molecular sieve assembly through an electromagnetic reversing valve, and the other compressed gas is connected to an gas-excited negative oxygen ion generator through a pressure gas interface.

3. The oxygen concentrator with negative oxygen ions according to claim 1, wherein a gas volume regulating valve or a limiting hole is also provided at the input end of the gas-excited negative oxygen ion generator to control the amount of gas input into the gas-excited negative oxygen ion generator.

4. The oxygen concentrator with negative oxygen ions according to claim 1, wherein the oxygen generator also includes an oxygen storage tank disposed in the device housing, the molecular sieve assembly inputs oxygen into the oxygen storage tank, and the oxygen storage tank is connected to an oxygen interface on one side of the device housing.

5. The oxygen concentrator with negative oxygen ions according to claim 1, wherein the device housing is provided with a display screen and a plurality of control buttons, and the display screen and the control buttons are connected to the control circuit board.

6. The oxygen concentrator with negative oxygen ions according to claim 4, wherein an oxygen pressure regulating valve and an oxygen sensor are provided at the output end of the oxygen storage tank; The oxygen sensor is arranged on the oxygen pipeline between the oxygen storage tank and the oxygen interface; The output end of the oxygen sensor is connected to the oxygen interface through a flow regulating valve, and the oxygen sensor is connected to the control circuit board.

7. A diffused oxygen system, comprising an oxygen generator, an intelligent controller arranged in the oxygen-demanding space, and a gas-excited negative oxygen ion generator, wherein the oxygen generator comprises an equipment housing, a control circuit board, a power supply device, a compressor, and a plurality of molecular sieve assembly arranged in the equipment housing, wherein the input end of the compressor is connected to an exhaust interface, and the input end of the compressor is connected to the oxygen-demanding space through the exhaust interface to extract gas, or extracts gas from the oxygen-demanding space and the external space simultaneously;
The output end of the compressor divides the compressed gas into two paths, one of which is connected to the molecular sieve assembly through an electromagnetic reversing valve, and the molecular sieve assembly inputs oxygen into the oxygen interface, and the oxygen interface inputs oxygen to the air outlet of the intelligent controller through a connecting pipeline; the other path is connected to the gas-excited negative oxygen ion generator through a connecting pipeline, and the gas-excited negative oxygen ion generator is arranged on the connecting pipeline outside or inside the oxygen-demanding space, and the output end of the gas-excited negative oxygen ion generator is connected to the air outlet of the intelligent controller, or the input end of the gas-excited negative oxygen ion generator is arranged at the air outlet of the intelligent controller, and the output end of the gas-excited negative oxygen ion generator is directly connected to the oxygen-demanding space.

8. The diffused oxygen system according to claim 7, wherein the oxygen generator is arranged outside the oxygen-demanding space, the exhaust interface of the compressor is connected to the oxygen-demanding space through an gas extraction pipeline, and all or part of the air in the oxygen-demanding space is extracted to produce oxygen, and external air is used to replenish the oxygen-demanding space for air balance.

9. The diffused oxygen system according to claim 7, wherein the oxygen generator is arranged in the oxygen-demanding space, and the exhaust interface of the compressor is connected to the oxygen-demanding space to extract gas, or is connected to the external space through an gas extraction pipeline and extracts gas from the oxygen-demanding space and the external space at the same time; the oxygen generator discharges nitrogen to the outside of the oxygen-demanding space through a nitrogen gas extraction pipeline.

10. The diffused oxygen system according to claim 7, wherein the intelligent controller is arranged in the oxygen-demanding space, including a sensor group for real-time monitoring of air state parameters and data exchange with a host computer; The sensor group is connected to or integrally arranged with the intelligent controller, and is used to detect the real-time oxygen concentration value and the real-time carbon dioxide concentration value in the oxygen-demanding space according to a preset period; When the real-time oxygen concentration value or the real-time carbon dioxide concentration value is abnormal, the operating parameters of the oxygen generator are queried and detected and an alarm is issued.
